SEQUENCE LISTING (2 sequences)

SEQ ID NO: 1 - DNA, 639 bp, Artificial
  Recombinant DNA
atgccatttgaattgccagcattgccgtatccgtatgatgcgcttgagccgcacatcgacaaagaaacgatgaacattcaccacacgaagcaccataacacatacgttacaaatttgaatgcggcgcttgaagggcatccggatttgcaaaacaaatcgctcgaagaattgctcagcaatttggaagcccttccggaaagcattcgcacggcggtgcgcaacaacggcggcggtcatgcaaaccactcgcttttctggacgattttgtcgccaaatggcggcggtgagccgacgggtgagctggctgaggcgatcaacaaaaaattcggcagcttcaccgcgtttaaagacgagttttcgaaagcagcggccggccgtttcggttctggctgggcatggcttgtcgtgaacaacggcgagctggaaattacgagcacgccgaaccaagactcgccgatcatggaaggcaaaacgccgattctcggcttggacgtttgggagcatgcgtactacttgaaataccaaaaccgccgtccggaatacattgccgcattctggaacattgtcaactgggacgaagtggcgaaacggtacagcgaagcgaaagcgaagctcgagcaccaccaccaccaccactga

SEQ ID NO: 2 - protein, 212 aa, Artificial
  fusion protein
MPFELPALPYPYDAELPHIDKEDMNIHHTKHHNTYVTNLNAALEGHPDLKNQSLEELLSNLEALPESIRTAVRNNGGGHANHSLFWTILSPNGGGEPTGELAEAINKKFGSFTASKDEFSKAAAGRFGSGWAWLVVNNGELEITSTPNQDSPIMEGKTPILGLDVWEHAYYLKYQNRRPEYIAAFWNIVNWDEVAKRYSEAKAKLEHHHHHH

(19) United States
(12) Patent Application Publication
(10) Pub. No.: US 2009/0311768 A1
(43) Pub. Date: Dec. 17, 2009

(54) Thermostable superoxide dismutase

(75) Inventor: Shir-Ly Huang, Taoyuan (TW)

Correspondence Address: J C PATENTS, 4 VENTURE, SUITE 250, IRVINE, CA, 92618, US

(73) Assignee: National Central University, Taoyuan (TW)

(21) Appl. No.: 12/316,724

(22) Filed: Dec. 15, 2008

(30) Foreign Application Priority Data
Jun. 13, 2008 (TW) 97122205

(51) Int. Cl. C12N 9/02; C12N 15/11

(52) U.S. Cl. 435/189; 536/23.2

(57) ABSTRACT

The present invention provides a thermostable superoxide dismutase from a thermophile. The thermostable superoxide dismutase that is able to catalyze the conversion of superoxide ions or radicals under the moderate to high temperature environments.

### CROSS-REFERENCE TO RELATED APPLICATION

This application claims the priority benefit of Taiwan application serial no. 97122205, filed Jun. 13, 2008. The entirety of the above-mentioned patent application is hereby incorporated by reference herein and made a part of this specification.

### BACKGROUND OF THE INVENTION

1. Field of the Invention

The present invention generally relates to an enzyme. In particular, the present invention relates to a thermostable superoxide dismutase, the fusion protein and the recombinant gene sequence thereof.

2. Description of Related Art

Functional enzymes have been widely used in industry manufacturing, medicine production and academic studies and are of high added-values. It becomes a trend to use enzymes as catalysts, instead of traditional catalysts containing heavy metals or harmful solvents, in green technology for environmental protection. However, heating processes or steps are common in industry manufacturing for increasing the reaction rate or the production, and most enzymes are not stable enough to endure heating.

Hence, it is highly desirable to keep enzyme proteins active and stable while being heated, for better and promising industrial applications.

### SUMMARY OF THE INVENTION

The present invention provides a fusion protein of a thermostable superoxide dismutase and its corresponding recombinant deoxyribonucleic acid (DNA) gene sequences. The obtained thermostable superoxide dismutase is able to catalyze the conversion of superoxide ions or radicals into harmless metabolites in the moderate to high temperature environments.

As embodied and broadly described herein, the obtained thermostable superoxide dismutase is able to catalyze in environments at temperatures of about 55-100° C. The thermostable superoxide dismutase of this invention was cloned from

Geobacillus kaustophilus ATCC8005.

As embodied and broadly described herein, the obtained thermostable superoxide dismutase is able to maintain their catalytic ability for at least 30 minutes in environments at temperatures of about 55-100° C. The thermostable superoxide dismutase of this invention reaches the highest catalytic activity at 65° C., while the melting temperature (Tm) range of the thermostable superoxide dismutase can reach about 91-96° C.

The thermostable superoxide dismutase provided by this invention is exceptionally useful in removing or subtracting harmful superoxides or radicals, and has valuable potentials in the industry of pharmaceuticals, cosmetics, food and/or biotechnology.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 shows the amino acid sequence for the fusion protein of the thermostable superoxide dismutase of this invention and its corresponding recombinant DNA gene sequence.

FIG. 2 shows the chromatographic diagram of the thermostable superoxide dismutase in the present invention.

FIG. 3 shows the specific activities & relative activities of the purified superoxide dismutase in the present invention after heating treatments for 30 minutes under different temperature ranges versus the temperatures of the heating treatments.

### DESCRIPTION OF THE EMBODIMENTS

Reference will now be made in detail to the present preferred embodiments of the invention, examples of which are illustrated in the accompanying drawings. Wherever possible, the same reference numbers are used in the drawings and the description to refer to the same or like parts.

Microorganisms can be grouped into different categories according to their temperature optima: hyperthermophiles (>80° C.), thermophiles (45-80° C.), mesophiles (20-45° C.), and psychrophiles (<20° C.).

Geobacillus kaustophilus ATCC8005 is a thermophilic bacterium grown in high temperature environments, with its optimal temperature at about 55° C. The fusion protein of the thermostable superoxide dismutase in the present invention was screened from Geobacillus kaustophilus ATCC8005.

The fusion protein of the thermostable superoxide dismutase of this invention can be screened and prepared according to the high-throughput screening method proposed by the inventor of this application (filed as Taiwanese Patent Application No. 095139059). The screening method is summarized as follows. At first, the crude cell extracts obtained from Geobacillus kaustophilus ATCC8005 were treated at 55, 65, 75, 85, 95 and 100° C. respectively for 30 min. The soluble sub-proteomes were obtained after ultracentrifugation to remove aggregated (inactive) proteins. After separating by one dimensional and two-dimensional-gel electrophoresis, the proteins were digested by trypsin in-gel. Another approach is the proteomes of which the proteins were digested without separation. The digested peptide mixtures were separated and the peptide sequenced by MALDI-Q-Tof MS or ESI-Q-Tof MS. The sequenced genome in the database allows us to accurately identify the target proteins (i.e. thermostable enzymes) in a high-throughput format. Following gene cloning technology to obtain the recombinant DNA sequence(s), the fusion protein(s) of the superoxide dismutase(s) is expressed in E. coli. system and the superoxide dismutase(s) is then separated and purified by using affinity column chromatography and gel filtration column chromatography.

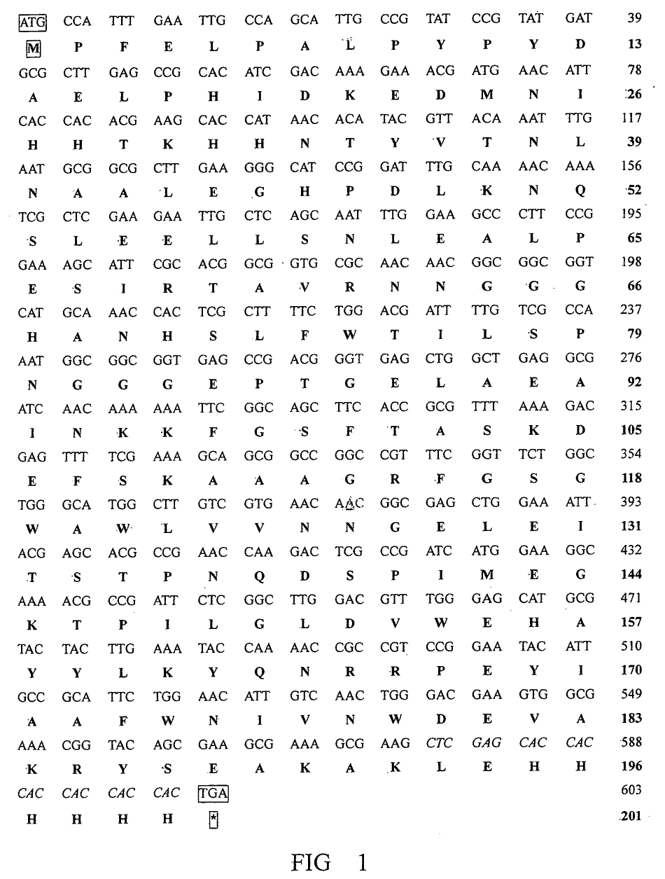

FIG. 1 shows the amino acid sequence for the fusion protein of the thermostable superoxide dismutase of this invention and its corresponding recombinant DNA gene sequence. The recombinant DNA for the fusion protein of the thermostable superoxide dismutase was constructed based on the superoxide dismutase gene of Geobacillus kaustophilus ATCC8005, and can be expressed in E. coli system to prepare the thermostable superoxide dismutase of this invention. In FIG. 1, ATG represents the initial code for the subsequent protein translation, while TGA represents the ending code for the subsequent protein translation. The gene sequence can be read by three letters and the translated amino acid is shown as a single letter underlying. The letters marked in italic represent the added nucleic acid sequence (that is, an extra eight-amino acid peptide fragment).

The recombinant DNA sequence for the fusion protein of the thermostable superoxide dismutase in the present invention is SEQ ID NO. 1 shown in the followings:

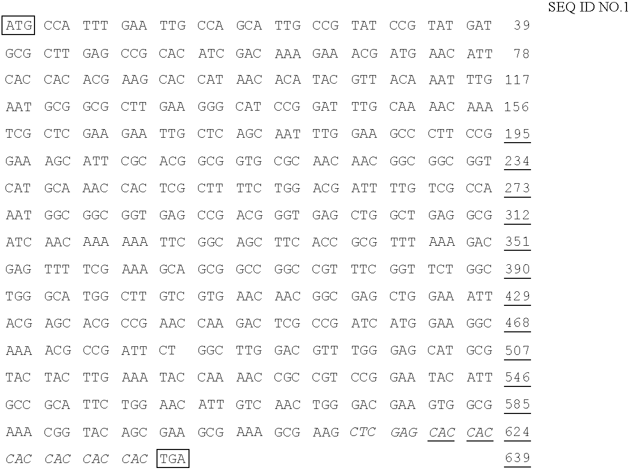

The preparation method generally employs the recombinant DNA sequence (SEQ ID No. 1) to obtain the fusion protein of the thermostable superoxide dismutase after separation and purification.

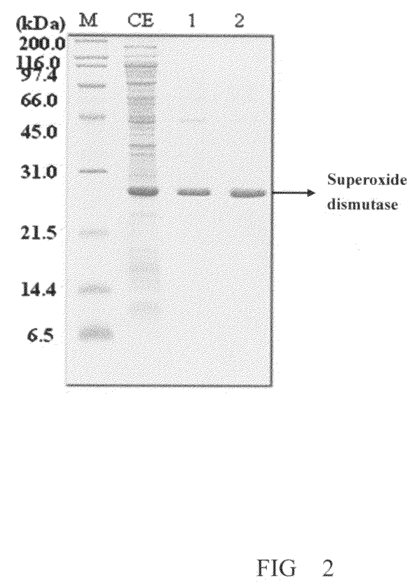

FIG. 2 shows the gel electrophoretic separation of the partially and purified thermostable superoxide dismutase in the present invention, wherein “M” represents the molecular standard marker, “CE” represents the crude protein extract obtained from E. coli before purification, “1” represents the sample after the first purification by affinity column chromatography, and “2” represents the sample after the second purification by gel filtration column chromatography. The above samples are analyzed by electrophoresis with 15% polyacrylamide gel and the results are shown in FIG. 2. The monomer of the superoxide dismutase in the present invention has a molecular weight of about 24 kDa, while the natural enzyme exists in homodimers with a molecular weight of about 47.1 kDa determined form gel filtration chromatography. When compared the analysis results of the crude extract shown in the second lane with those of the third and fourth lanes, the purity of the obtained superoxide dismutases after two column chromatography in the present invention is relatively high.

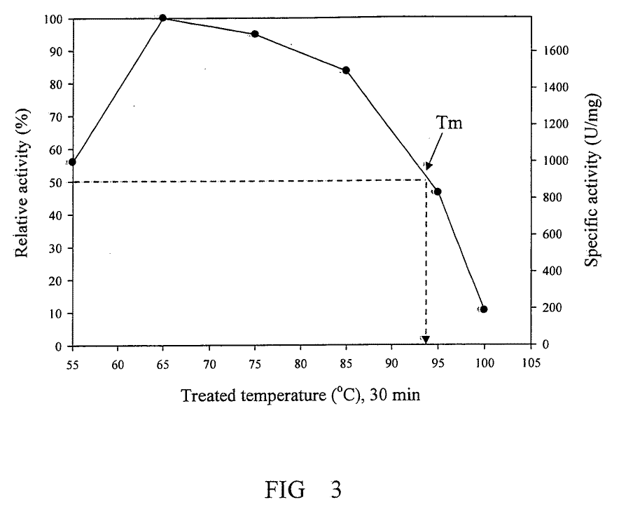

FIG. 3 shows the specific activities & relative activities of the purified superoxide dismutase in the present invention after heating treatments for 30 minutes under different temperature ranges versus the temperatures of the heating treatments. The X axis represents the temperature of heating treatments, the left Y axis represents the relative activity of the treated enzyme, and the right Y axis represents the specific activity of the treated enzyme. It is found that the thermostable superoxide dismutase of the present invention reaches the highest catalytic activity at about 65° C. In this embodiment, after treating under 65° C. for 30 minutes, the treated enzyme has a specific activity of 1786 U/mg. In general, the suitable reaction temperature for the thermostable superoxide dismutase in the present invention ranges between about 60° C. to 75° C., for example.

If setting the highest specific activity of the enzyme (herein the enzyme treated under 65° C. for 30 minutes) as 100%, the temperature where the enzyme activity reaches 50% is defined as the melting temperature (Tm) of the enzyme. Hence, from FIG. 3, Tm of the enzyme in this embodiment is set at about 94° C. According to the experimental data, Tm of the superoxide dismutase in the present invention is about 91-96° C.

In general, the purified superoxide dismutase in the present invention can keep the catalytic activity under the environments of about 55-100° C. for at least 30 minutes. The appropriate reaction temperature range of the superoxide dismutase in the present invention is about 60-75° C., preferably at 65° C. The melting temperature of the superoxide dismutase in the present invention can be as high as 91-96° C.

Therefore, the obtained superoxide dismutase of the present invention is thermostable and remains catalytic activity under the medium to high temperature environments. The purified superoxide dismutase in the present invention can keep the catalytic activity under the environments of about 55-100° C. for at least 30 minutes, with a specific activity of about 186-1696 U/mg. Taking heating treatment for 30 minutes as an example, the thermostable superoxide dismutase of the present invention reaches the highest catalytic activity at about 65° C., while Tm of the superoxide dismutase in the present invention is about 91 -96° C.

The thermostable superoxide dismutase of the present invention is the first superoxide dismutase obtained from the thermophiles Bacillus and the related genus Geobacillus. Compared with the superoxide dismutases from other thermophiles, such as the superoxide dismutases from Rhodothermus sp. XMH10 having the highest activity at 65° C. and Tm of 70-80° C. after heating for 30 minutes (Wang et al., 2008), or the superoxide dismutases from Thermomyces lanuginosus having the best activity at 55° C. and Tm of 70-75° C. after heating for 30 minutes (Li et al., 2005), the superoxide dismutase of the present invention has exceptionally better thermostability.

It is noted that the superoxide dismutase has the ability of catalyzing the destruction of harmful superoxides or radicals, which renders the thermostable superoxide dismutase of the present invention very useful in the industry of pharmaceuticals, cosmetics, food, biotechnology and environmental protection. As the thermostable superoxide dismutase of the present invention has superior thermostability, the enzyme proteins are not easily degenerated and its functioning period is relatively longer in the applied products.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What claimed is:

1. A fusion protein, which corresponds to a recombinant deoxyribonucleic acid (DNA) sequence of SEQ ID NO. 1.

2. The fusion protein of claim 1, wherein said fusion protein comprises a thermostable superoxide dismutase.

3. The fusion protein of claim 2, wherein said thermostable superoxide dismutase remains a catalytic activity under environments of about 55-100° C. for at least 30 minutes, while said thermostable superoxide dismutase has a specific activity of about 186-1696 U/mg under the environments of about 55-100° C.

4. The fusion protein of claim 2, wherein said thermostable superoxide dismutase has the highest catalytic activity at about 65° C.

5. The fusion protein of claim 2, wherein a melting temperature of said thermostable superoxide dismutase is about 91-96° C.

6. A recombinant deoxyribonucleic acid (DNA) sequence fragment, comprising a sequence of SEQ ID NO. 1.
